(19) United States
(12) Patent Application Publication
(10) Pub. No.: US 2025/0177998 A1
(43) Pub. Date: Jun. 5, 2025

(54) BLOOD PROCESSING SYSTEMS AND METHODS

(71) Applicant: Kyriacos Matsis, Nicosia (CY)

(72) Inventor: Kyriacos Matsis, Nicosia (CY)

(21) Appl. No.: 18/968,724

(22) Filed: Dec. 4, 2024

Related U.S. Application Data
(60) Provisional application No. 63/605,941, filed on Dec. 4, 2023.

(51) Int. Cl. B04B 7/12; B04B 13/00; C12N 5/00; G01N 33/49

(52) U.S. Cl. CPC B04B 7/12; B04B 13/00; C12N 5/544; G01N 33/491; B04B 2013/006

(57) ABSTRACT

A blood sample processing system and method for processing of umbilical cord blood is disclosed. The processing system is configured to ensure reliable processing of the blood sample from collection to testing, and includes a blood bag housing device, an isolation device, and a cryopreparation device. The blood bag housing device provides a secure and reliable housing for a blood sample bag while in the bucket of a centrifuge. The isolation device provides a simple and repeatable system for the isolation of the blood components separated during centrifugation. The isolated blood component sample from the isolation device is transferred into a cryobag and is ready for the administration of the cryoprotectant. The cryopreparation device provides a system with controlled temperature, ensuring precise conditions for the administration of cryoprotectant to the isolated blood component sample from the isolation device.

### CROSS-REFERENCE TO RELATED APPLICATIONS

This application claims the benefit of priority to U.S. Provisional Application 63/605,941 filed Dec. 4, 2023, the entire disclosure of which is hereby incorporated by reference.

### BACKGROUND OF THE INVENTION

The present subject matter relates generally to the separation and isolation of certain components of blood product. Specifically, the disclosure pertains to devices designed for processing umbilical cord blood samples to produce stem leukocyte-rich (commonly known as buffy coat) units for long-term storage, intended for potential medical treatment or use. The process involves three key stages: blood component separation, leukocyte concentrate or buffy coat isolation, and preparation for cryostorage or cryoprotectant administration.

Centrifugation is a necessary process for the separation of blood components or, more specifically, umbilical cord blood components including plasma, red blood cells, and white blood cell concentrates (white blood cell concentrates often being referred to as “buffy coat”). However, this process often poses challenges in laboratories. The relative centrifugal force exerted on the centrifuge bucket and its contents during blood centrifugation can exceed 5,000 Newtons, leading to the crumpling up of the blood bag and not allowing proper separation of the blood components, or spillage and damage to the processing set or blood bags inside the centrifuge's bucket. These issues pose significant concerns as spilled blood can carry infectious risks for operators and necessitates time-consuming preparation of the samples for centrifugation or sterilization of affected surfaces in case of bag damage or spillage. Most importantly, bag damage and blood spillage results in the loss of valuable blood components, rendering the sample unsuitable for further processing and adversely impacting the quality of laboratory services.

To minimize these challenges, lab operators traditionally use sponges or other supporting material around the blood bag and prepare it for centrifugation, but such solutions do not resolve the problem completely. Therefore, a need exists for a system that consistently and effectively allows for the separation of blood components during centrifugation while minimizing loss of blood samples.

Additionally, isolating the necessary components for testing in different containers from the single container often requires operator judgment to visually distinguish the different regions of blood components. If an operator's judgment is imprecise or includes any inaccuracy, some mixture of previously separated components in the new container will occur, which in turn compromises the quality of the final product to be stored and results in testing inefficiencies. A need thus exists for an easy, precise, effective, and cost-efficient system for isolating separated blood components, while also minimizing the amount of operator judgment involved and the training time needed for the operators.

Still further, once the components are effectively isolated in separate containers, the leukocyte concentrate or buffy coat must be maintained within precise temperature ranges to properly preserve the sample during the subsequent steps, including the administration of cryoprotectant, until it is placed in cryostorage. Traditionally, blood samples are kept on ice during cryoprotectant administration and stored in an insulated container. However, this approach can cause unexpected temperature spikes, leading to unpredictable outcomes that may compromise the sample. Dimethylsulfoxide (DMSO) is commonly used as a cryoprotectant, but its administration also demands precise temperature control for optimal effectiveness. Therefore, there is a need for a cryopreparation device for blood component samples that ensures consistent and predictable temperature regulation during DMSO administration.

Accordingly, there is also a need for an overall integrated processing system capable of solving all of the above needs.

### BRIEF SUMMARY OF THE INVENTION

To meet the needs described above and others, the present disclosure provides an example blood processing system including a blood bag housing device, an isolation device, and a cryopreparation device. In some embodiments, the isolation device is arranged for manual operation or for automated operation. The blood processing system is used for the processing of any blood sample, and each device is utilized to facilitate the production of high-quality cellular therapy products at various stages of the umbilical cord blood processing.

In a first aspect of the present disclosure, which may be combined with any other aspect listed herein unless specified otherwise, a blood bag housing device of a blood processing system is disclosed. The blood bag housing device comprising a main body including a front portion and a back portion spaced from the front portion by a cavity, wherein the back portion includes a curved surface at a top of the main body, and wherein a bottom of the main body includes one or more pathways. The blood bag housing device also includes a retainer cover removably couplable to the main body, and the main body is configured to seat in a bucket of the centrifuge.

In a second aspect of the present disclosure, which may be combined with the first aspect in combination with any other aspect listed herein unless specified otherwise, a back pocket in the back portion is configured to house at least one tubing coupled to the top of the at least one whole blood bag when the retainer cover is affixed to the back pocket, and a front pocket in the front portion is configured to house at least one tubing coupled to the bottom of the at least one whole blood bag.

In a third aspect, which may be combined with the first aspect in combination with any other aspect listed herein unless specified otherwise, at least one of the one or more pathways is configured to receive at least one of the at least one tubing housed in the back pocket.

In a fourth aspect, which may be combined with the first aspect in combination with any other aspect listed herein unless specified otherwise, the blood bag housing device is configured to be submerged in water during centrifugation.

In a fifth aspect, which may be combined with the first aspect in combination with any other aspect listed herein unless specified otherwise, the retainer cover includes an aperture.

In a sixth aspect, which may be combined with the first aspect in combination with any other aspect listed herein unless specified otherwise, the front pocket or the back pocket may be configured to house at least one satellite bag during centrifugation.

In a seventh aspect, which may be combined with the first aspect in combination with any other aspect listed herein unless specified otherwise, the retainer cover comprises at least one tabbed surface that is received by a slotted opening in the main body.

In an eighth aspect, which may be combined with any other aspect or embodiment listed herein unless specified otherwise, an isolation device of a blood processing system is disclosed. The isolation device comprises a frame, a blood bag neck holder mounted to the frame, the blood bag neck holder comprising an upper holder rod configured to receive a top opening of a blood bag, a blood bag tensioner secured to the frame, the blood bag tensioner comprising a lower holder rod configured to receive a bottom opening of a blood bag, and an isolation plate arrangement mounted to the frame. The isolation plate arrangement comprises a front plate and a back plate movable relative to each other. The isolation plate arrangement is configured to at least partially house the blood bag between the front plate and the back plate when the blood bag is secured to the blood bag neck holder and the blood bag tensioner.

In a ninth aspect, which may be combined with the eighth aspect in combination with any other aspect listed herein unless specified otherwise, the back plate is secured to the frame and includes a threaded bolt extending forwardly from the back plate and through an opening in the front plate, and the isolation plate arrangement includes a pressure knob that engages the threaded bolt and moves the front plate relative to the back plate.

In a tenth aspect, which may be combined with the ninth aspect in combination with any other aspect listed herein unless specified otherwise, the front plate further comprises a plurality of alignment holes disposed around a perimeter of the front plate, and the back plate further comprises a plurality of alignment rods configured to slidably engage the alignment holes to align the front plate and the back plate during operation of the isolation device.

In an eleventh aspect, which may be combined with the ninth aspect in combination with any other aspect listed herein unless specified otherwise, the back plate is secured to a support plate through which an axle extends, a gear and a vertical adjustment knob are affixed to the axle adjacent the support plate, and the gear engages a geared track attached to the frame such that rotation of the gear translates to a vertical displacement of the back plate.

In a twelfth aspect, which may be combined with the eighth aspect in combination with any other aspect listed herein unless specified otherwise, the back plate further comprises at least one spacer configured to provide a consistent spacing between the front plate and the back plate as the at least one torque-limited pressure knob is adjusted.

In a thirteenth aspect, which may be combined with the eighth aspect in combination with any other aspect listed herein unless specified otherwise, the isolation plate arrangement comprises one or more rotors configured to move at least one of the front plate and the back plate, at least one clamp actuator configured to move a clamp toward one of the front plate and the back plate such that the one of the front plate and the back plate are secured in position, and a sensor configured to detect blood components in the blood bag disposed between the front plate and the back plate. The sensor and at least one rotor are controlled by a control unit.

In a fourteenth aspect, which may be combined with any other aspect listed herein unless specified otherwise, a cryopreparation device of a blood processing system is disclosed. The cryopreparation device comprises a base including a cavity stack compartment, a cover, a cavity stack compartment in an interior of the base, and a cavity stack disposed in the cavity stack compartment. The cavity stack includes a plurality of cavity plates configured to be stacked within the cavity stack compartment to seat a cryobag. A first cavity plate on a top side of the cavity stack comprises a compartment configured to enclose the cryobag and at least one tube channel configured to receive a cryoprotectant tube, and a second cavity plate disposed below the first cavity plate comprises a fluid pathway configured to allow the flow of a cooling fluid in the interior of the base.

In a fifteenth aspect, which may be combined with the fourteenth aspect in combination with any other aspect listed herein unless specified otherwise, the cryopreparation device is configured to maintain a temperature of the cryobag at 4° C.±0.1° C.

In a sixteenth aspect, which may be combined with the fifteenth aspect in combination with any other aspect listed herein unless specified otherwise, the cryopreparation device further comprises a temperature sensor configured to sense a temperature of the cryobag, and the cryopreparation device is configured to circulate cooling fluid in the interior of the base if the temperature of the cryobag reaches 4.1° C. until it drops to a minimum 3.9° C. where the circulation of cooling fluid stops.

In a seventeenth aspect, which may be combined with the fourteenth aspect in combination with any other aspect listed herein unless specified otherwise, the cryopreparation device further comprises a motor configured to pulsate fluid inside the cryobag.

In an eighteenth aspect, which may be combined with the seventeenth aspect in combination with any other aspect listed herein unless specified otherwise, a mixer is coupled to the motor and configured to evenly mix fluid inside the cryobag.

In a nineteenth aspect, which may be combined with the fourteenth aspect in combination with any other aspect listed herein unless specified otherwise, the cryopreparation device further comprises an aluminum plate disposed on an interior of the cover.

In a twentieth aspect, which may be combined with the fourteenth aspect in combination with any other aspect listed herein unless specified otherwise, the cavity stack is affixed to the base by a plurality of fasteners through a plurality of holes disposed around a perimeter of the cavity stack and a perimeter of the cavity stack compartment.

An object of the invention is to provide a solution for secure and reliable housing of a whole blood bag containing a blood sample during centrifugation.

Another object of the invention is to provide a solution for isolating desired components of the whole blood sample separated by the centrifugation process.

Another object of the invention is to provide a solution that ensures optimal temperature conditions for the isolated blood components, enabling successful cryoprotectant administration without compromising the sample.

A further advantage of the invention is that it provides a full system for the separation, isolation, and preparation for cryopreservation of a blood sample.

An advantage of the invention is that it provides a simple, reliable, and repeatable system for the separation, isolation, and preparation for cryopreservation of a blood sample.

Another advantage of the invention is that it reduces the incidence of lost or destroyed blood sample, which cannot be replaced.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1A illustrates components of an exemplary blood processing system.

FIG. 1B is a front view of a blood processing set to be used in the system of the present disclosure.

FIG. 1C is a detailed view of whole blood bag illustrating separated blood components after use of the blood bag housing device according to the present disclosure.

FIGS. 2A to 2D are front perspective, back perspective, bottom, and right side views, respectively, of an embodiment of a blood bag housing device according to the present disclosure.

FIGS. 3A and 3B are right side and back perspective views, respectively, of the blood bag housing device of FIGS. 2a to 2d with a blood bag installed according to the present disclosure.

FIG. 4A is a front perspective view of an embodiment of an isolation device in a closed configuration and in a manual configuration according to the present disclosure.

FIG. 4B is a front perspective view of the isolation device of FIG. 4a in a closed configuration according to the present disclosure.

FIG. 4C is a front perspective exploded view of the isolation device of FIG. 4a according to the present disclosure.

FIG. 4D is a front perspective view of an embodiment of a blood bag neck holder of the isolation device of FIG. 4a according to the present disclosure.

FIG. 4E is a front perspective view of an embodiment of a blood bag tensioner of the isolation device of FIG. 4a according to the present disclosure.

FIG. 4F is a front perspective view of a manual isolation plate arrangement of the isolation device of FIG. 4A according to the present disclosure.

FIG. 5 is a front perspective view of an automated isolation plate arrangement of the isolation device of FIG. 4A in an automated configuration according to the present disclosure.

FIG. 6A is a front perspective view of an embodiment of a cryopreparation device in a closed configuration according to the present disclosure.

FIG. 6B is a front perspective view of the cryopreparation device of FIG. 6A in an open configuration according to the present disclosure.

FIG. 6C is a front perspective exploded view of the cryopreparation device of FIG. 6a according to the present disclosure.

FIG. 6D is a front perspective view of an internal arrangement of a cavity plate stack of the cryopreparation device of FIG. 6A according to the present disclosure.

### DETAILED DESCRIPTION OF THE INVENTION

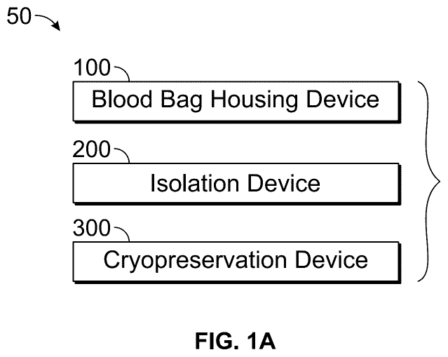

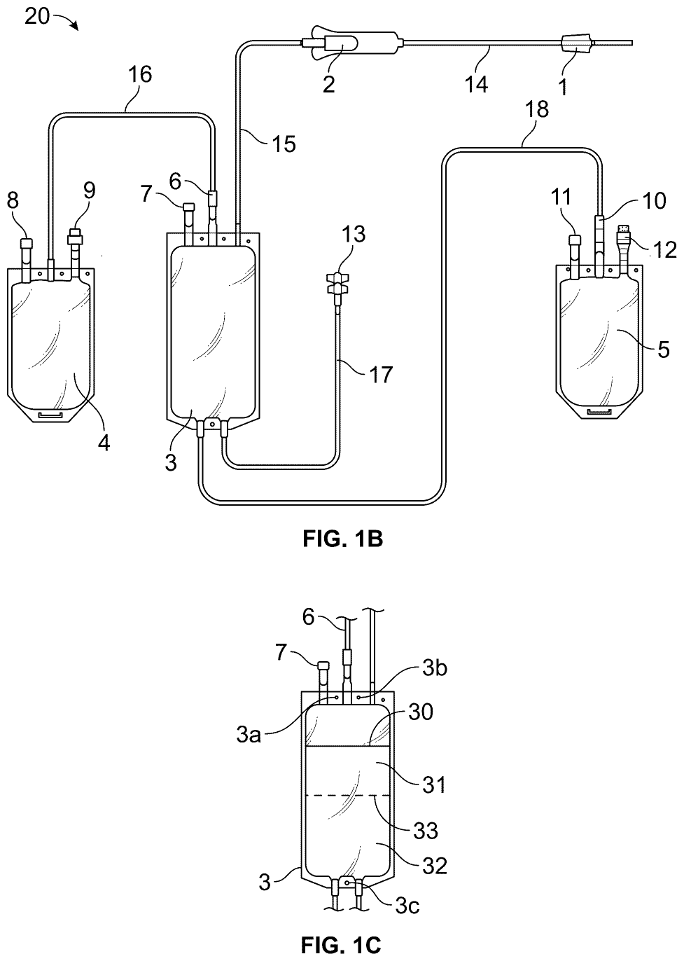

FIGS. 1A-6D illustrate an example blood processing system 50 including a blood bag housing device 100, an isolation device 200, and a cryopreparation device 300 that may be used for the processing of any blood sample to, for example, produce or prepare a cellular therapy product for cryopreservation. In one example, the processing system 50 is designed to isolate the leukocyte-rich layer (buffy coat), which constitutes the cellular therapy product, and prepare it for cryostorage (cryopreservation), ensuring its readiness for potential future medical use.

The blood bag housing device 100 achieves separation of blood components through centrifugation and is designed to hold the blood bag 3 in an upright position during centrifugation. The isolation device 200 facilitates the precise isolation of the centrifuged blood components and subsequently transfer each component to a separate compartment or blood bag. The cryopreparation device 300 administers a cryoprotectant in controlled conditions to ensure preparation of cellular therapy products for cryopreservation.

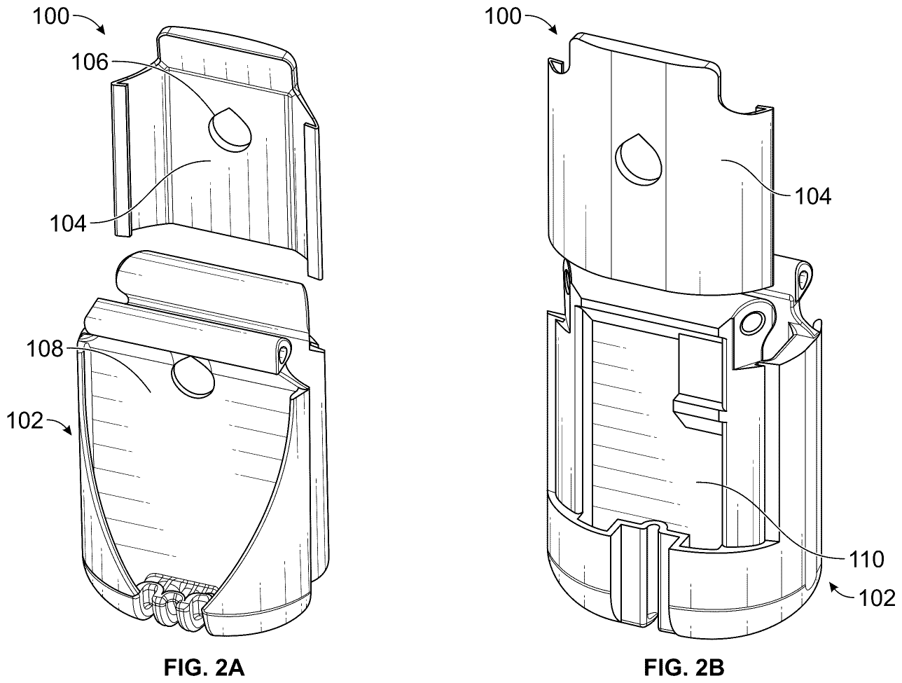

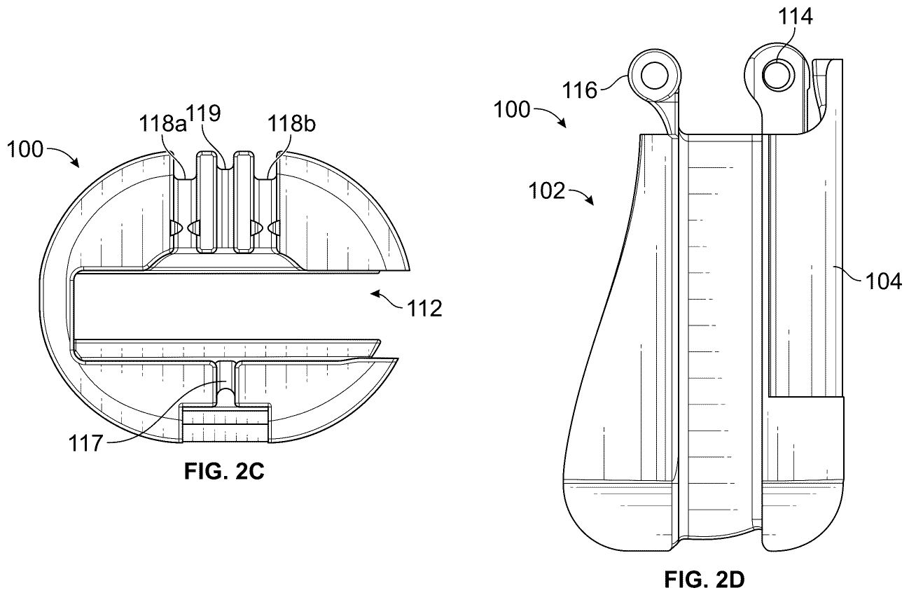

FIGS. 1B and 1C illustrate an example embodiment of a blood processing set 20 to be used in the system 50 of the present disclosure. The blood processing set 20 achieves sample volume reduction and is configured to be used for the processing of any blood sample, including umbilical cord blood. The processing set 20 also facilitates the collection of a leukocyte-enriched layer, also referred to as a buffy coat layer, after centrifugation and the isolation of the buffy coat in a whole blood bag 3. The blood processing set 20 further allows for the isolation of plasma and red blood cells from the blood sample, and the transfer of these blood components to a satellite plasma bag 4 and a satellite red blood cell bag 5, respectively. The processing set 20 may be used in manual processing procedures, the processing set 20 being a closed system which protects the blood sample from being contaminated during processing and which can be used with the system 50 and associated device disclosed in FIGS. 2A to 6D.

Referring to FIG. 1B, the blood processing set 20 includes three bags: the whole blood bag 3, the satellite plasma bag 4, and the satellite red blood cell bag 5. Blood is drawn into the whole blood bag 3 by an intravenous (IV) port 1 through a first IV tubing set 14, a blood sample filter 2, and a second IV tubing set 15 connected to the top end of the whole blood bag 3, each thereby fluidly connected to the whole blood bag 3.

The whole blood bag 3 is fluidly connected to the satellite plasma bag 4 by a plasma tubing set 16 through a whole blood breakaway cannula 6 disposed on a top end of the whole blood bag 3. The whole blood bag 3 is also fluidly connected to the satellite red blood cell bag 5 by red blood cell tubing set 18 which is connected to a bottom end of the whole blood bag 3. A red blood cell breakaway cannula 10 is positioned at a top end of the satellite red blood cell bag 5.

An extra tubing set 17 with a seal 13 on one end is also fluidly connected to the bottom of the whole blood bag 3. A plurality of sampling ports 7, 8, 9, 11, 12 are disposed on the top ends of the bags 3, 4, 5 and configured to allow injections to be added to the samples or for further samples to be extracted from the samples.

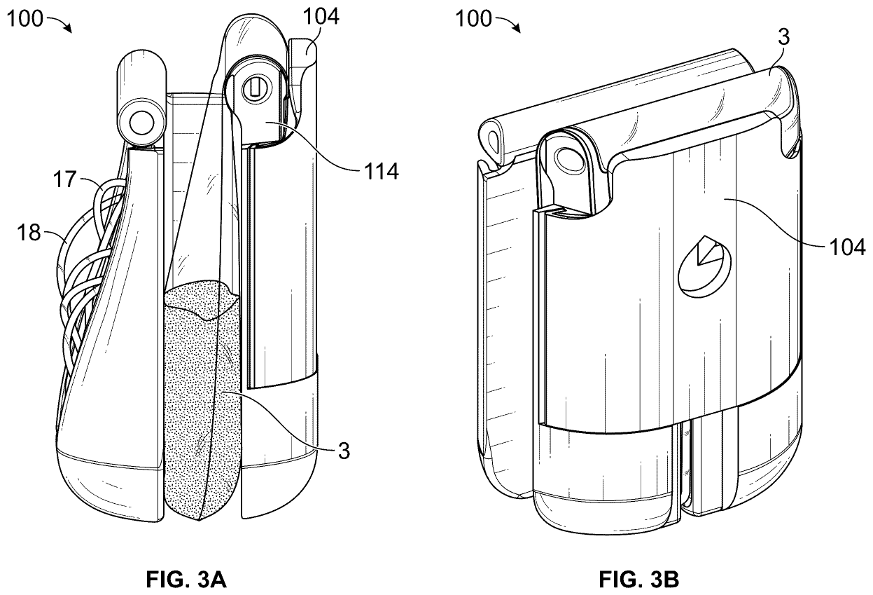

As illustrated best in FIGS. 1C and 3C, the processing set 20 is positioned within the blood bag housing device 100 during centrifugation, where the components of the blood sample are effectively separated into three different layers within the whole blood bag 3. After centrifugation, the whole blood bag 3 has three primary components below a fluid level 30: a plasma component 31 separated from a red blood cell component 32 by a buffy coat line 33. The whole blood bag 3 also includes two upper openings 3a, 3b and a bottom opening 3c along outer margins thereof, which will be used for ensuring proper tension of the bag in the isolation device 200 described later on.

FIGS. 2A to 3B illustrate a blood bag housing device 100 of the blood processing system 50. The blood bag housing device 100 is configured to provide a secure and stable positioning for the whole blood bag 3 during the centrifugation process, ensuring optimal separation of the blood components 30, 31, 32 and eliminating the risk of damage to the whole blood bag 3. Although a preferred embodiment is shown in the drawings, the blood bag housing device 100 may be configured with various dimensions to accommodate various processing sets or blood bags and various centrifuge bucket sizes.

The blood bag housing device 100 comprises two primary components: a main body 102 and a retainer cover 104. The main body 102 has an elongated vertical shape with a front portion spaced from a back portion by a central cavity 112. During use, the whole blood bag 3 is positioned in the central cavity while the satellite plasma bag 4 and the satellite red blood cell bag 5 are positioned in a front pocket 108 in the front portion and a back pocket 110 in the back portion. The front portion includes a front planar surface having a curved portion 116 at an upper end thereof, and the back portion includes a back planar surface having a curved portion 114 at an upper end thereof.

The blood bag housing device 100 has a user-friendly, ergonomic arrangement which facilitates relatively effortless assembly and disassembly in a matter of seconds by coupling or decoupling the retainer cover 104 from the back portion of the main body 102 above the back pocket 110 a sliding slotted connection. The retainer cover 104 includes tabbed surfaces on the left and right sides thereof, and the main body 102 includes slotted openings to receive the tabbed surfaces slidably on the left and right sides of the back pocket 110.

The main body 102 and the retainer cover 104 are constructed from food-grade, non-toxic materials that are resistant to liquid absorption. The selection of materials enables effective cleaning and decontamination (such as sterilization) of the blood bag housing device 100, thus allowing safe reuse by eliminating the risk of contaminating future blood samples or endangering the operator by contact with blood particles.

During use, the whole blood bag 3 containing a whole blood sample is inserted into the central cavity 112 of the blood bag housing device 100. An upper segment 3a of the whole blood bag 3 is folded and firmly positioned at the top of the curved surface 114 on the upper side of the main body 102 as shown in FIG. 3A. The top part of the whole blood bag 3 is folded along the back side of the blood bag housing device 100, above the back pocket 110, where there is a space to accommodate the injection port 7 and/or sampling port of the whole blood bag 3.

The retainer cover 104 is slid into the main body 102, which causes the whole blood bag 3 to be held tightly and securely in place, preventing the whole blood bag 3 from moving during centrifugation. The curved surface 114 on the upper side of the main body 102 safeguards the structural integrity of the processing set 20 throughout the centrifugation procedure. The back pocket 110 and the front pocket 108 securely hold the components other than the whole blood bag 3 of the processing set 20 during centrifugation, such as the satellite red blood cell bag 5 and the satellite plasma bag 4.

Upper tubings 15, 16 and lower tubings 17, 18 connected to the whole blood bag 3 are resiliently fitted through the arcuate pathways 117, 118a, 118b, 119 on the bottom of the main body 102 as shown in FIG. 2C. The outer arcuate pathways 118a, 118b include inwardly projecting tabs to further secure the tubings therein. The central arcuate pathways 117, 119 allow the upper tubings from the top of the whole blood bag 3 to extend over the curved portions 114, 116 and down through the front pocket 108. All tubings are then placed in the pockets 108, 110 along with the satellite plasma bag 4 and the satellite red blood cell bag 5 of the processing set 20. The blood bag housing device 100 is configured to ensure a near-perfect fit into most standard blood bag centrifuge buckets, and the size and dimensions of the blood bag housing device 100 may be adjusted in alternative embodiments to fit in any centrifugation bucket, facilitating seamless integration during the centrifugation process. The retainer cover 104 and planar surfaces of the main body 102 may include apertures 106 to enable an operator to confirm proper placement of the components in the back pocket 110 before centrifugation.

The blood bag housing device 100 secures the whole blood bag 3 in a stable position during centrifugation, thus allowing optimum centrifugation to take place.

The blood bag housing device 100 is configured to allow the insertion of sterile water into the central cavity 112 where the whole blood bag 3 is placed while in the centrifuge bucket by providing structural resilience for the whole blood bag 3 while suspended in sterile water. In some embodiments, sterile water is inserted into the centrifuge bucket up to the level of the blood sample in the whole blood bag 3. This procedure safeguards the bag from potential damage caused by the high force exerted by the blood sample against the internal walls of the whole blood bag 3 during centrifugation. The water is distributed evenly around the whole blood bag 3 so that any force applied by the blood within the whole blood bag 3, directed towards the outer walls of the whole blood bag 3 (which is a primary cause of potential bag damage during centrifugation), is counteracted by an equal and opposite force from the water surrounding the bag. Surrounding the bag uniformly with sterile water distributes the forces acting towards the outer walls of the whole blood bag 3, which reduces the incidence of failure of the bag walls. Testing has revealed that use of the blood bag housing device 100 at centrifuge speeds even up to double those required for most blood samples resulted in no bag damage during centrifugation.

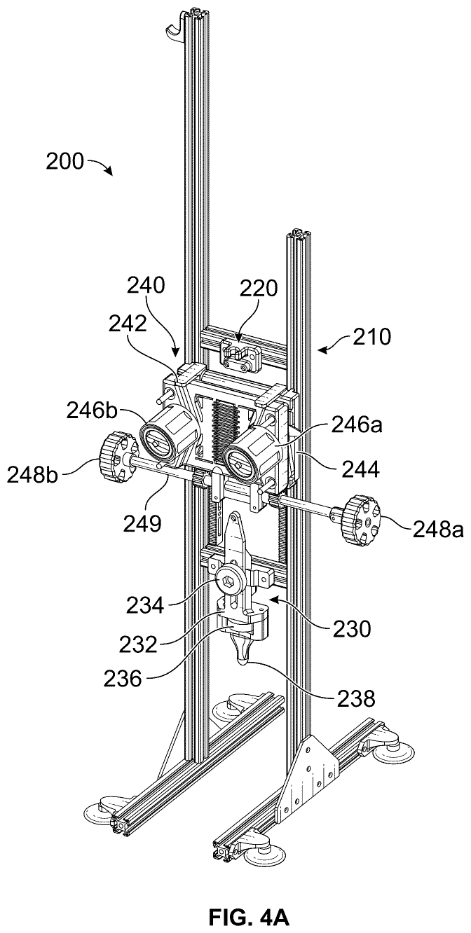

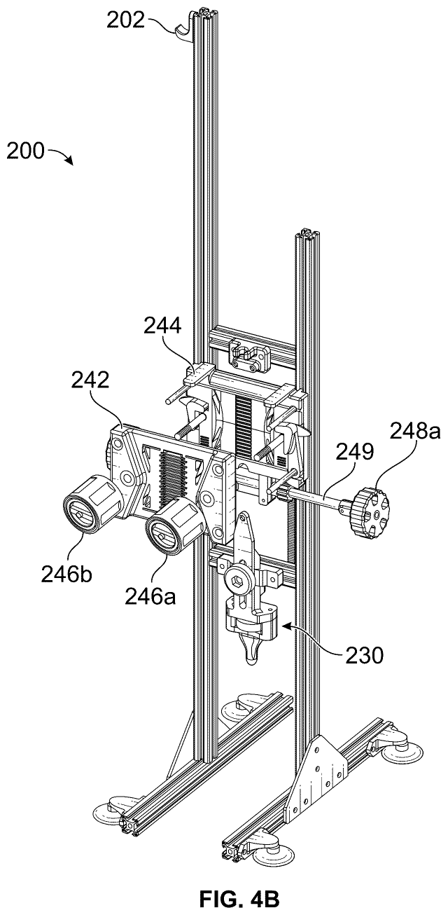

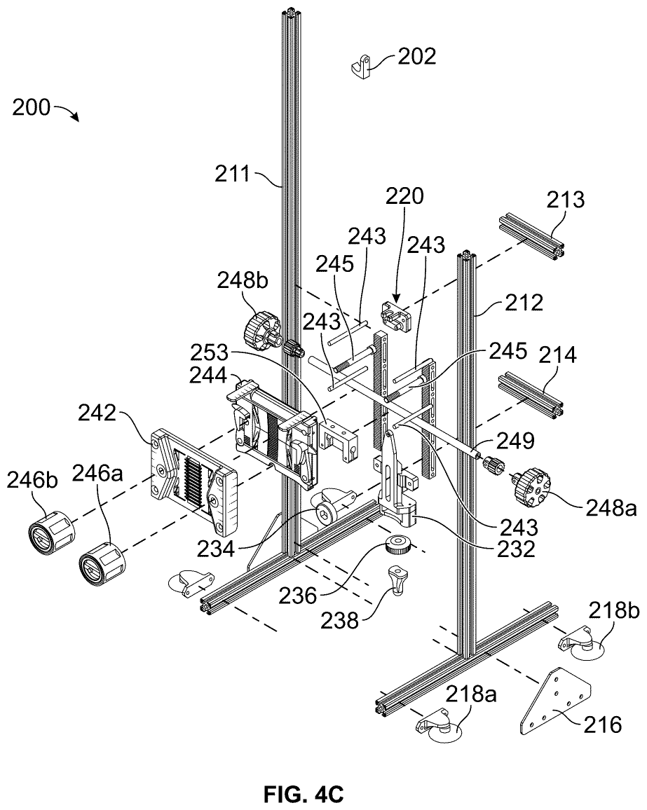

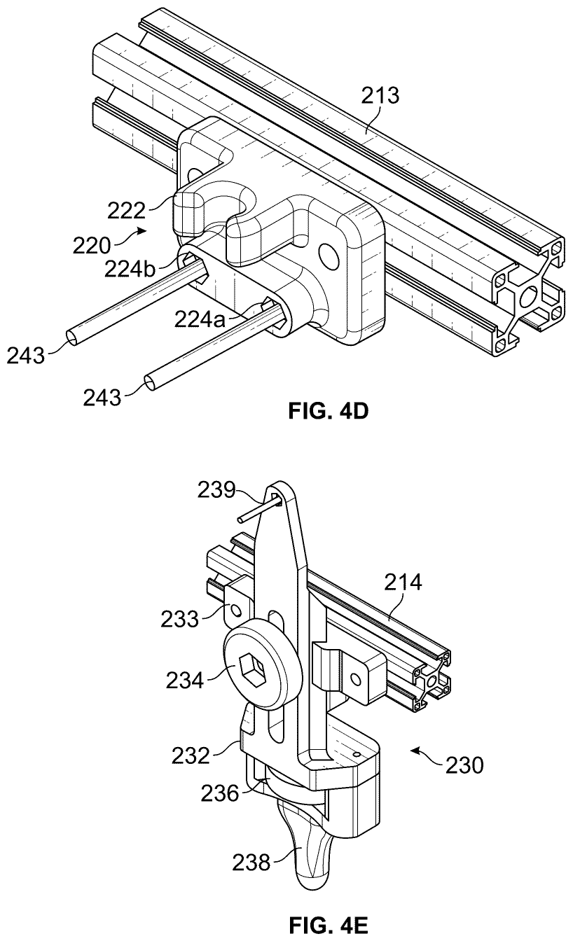

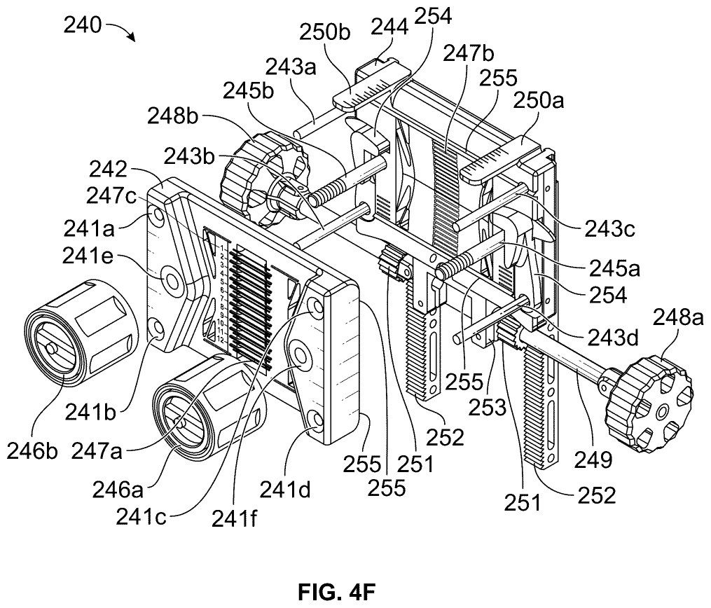

FIGS. 4A to 4F illustrate an isolation device 200 of the blood processing system 50. Once separation of the components within the whole blood bag 3 is complete, the processing set 20 is manually transferred to the isolation device 200. The isolation device is designed to facilitate the transfer and isolation of the blood components or any combination thereof into the three separate bags of the processing set 20. Specifically, the isolation device 200 transfers the plasma into the satellite plasma bag 4, the red blood cells into the satellite red blood cell bag 5, and leaves the remaining white blood cells, or buffy coat, in whole blood bag 3. While the isolation device 200 is described below with reference to the whole blood bag 3 and the processing set 20, it is understood that any blood bag of the same dimensions having the components therein separated via centrifugation may be used with the isolation device 200.

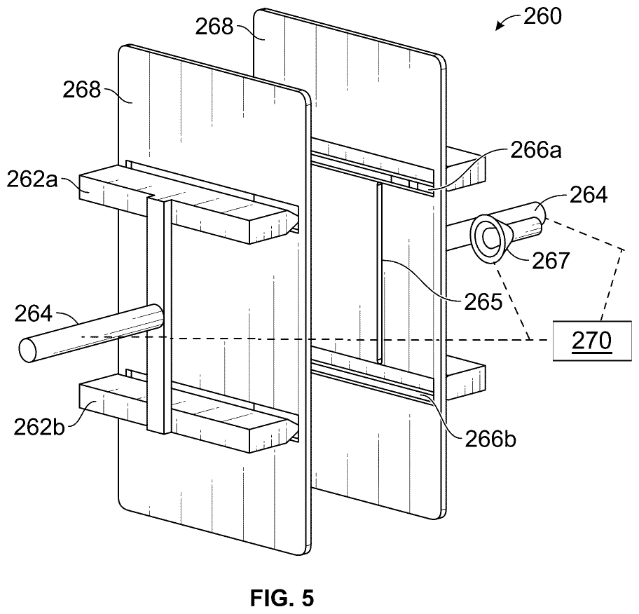

The isolation device 200 primarily comprises a frame 210, a blood bag neck holder 220, a blood bag tensioner 230, and an isolation plate arrangement 240. The embodiment of FIGS. 4A to 4F illustrate a manual isolation place arrangement 240, although an automatic isolation place arrangement 260 as described and shown in FIG. 5 below may also be used.

The frame 210 generally includes extruded components 211, 212, 213, 214 which provide structure for the mounting of the blood bag neck holder 220, the blood bag tensioner 230, and the isolation plate arrangement 240. Gussets 216 along the base components provide a rigid attachment of the extruded components 211, 212, 213, 214 thereto. The base components may also include feet or suction cups 218a, 218b facing the ground and configured to provide resilience to the isolation device 200 during operation.

The whole blood bag 3 is secured to the isolation device 200 in the same or equivalent upright position that it maintained during centrifugation. Referring to FIGS. 1B, 1C, 4D and 4E, the whole blood bag 3 is positioned such that the openings 3a and 3b on the top of whole blood bag 3 receive rods 243 and further by inserting the blood bag tensioner hook 239 of the blood bag tensioner 230 into the bottom opening 3c of the whole blood bag 3. The blood bag neck holder 220 includes a tubing holder 222 configured to hold tubing coming off of the whole blood bag 3 in place during the isolation process.

The blood bag tensioner 230 includes a tensioner body 232 which is slidably seated on a tensioner rail 233. A set screw knob 234 holds the tensioner body 232 in place on the tensioner rail 233 or can be loosened in order to create or reduce tension in the whole blood bag 3 by sliding the tensioner body 232 along the tensioner rail 233 in the desired direction. The blood bag tensioner 230 also includes a fine-tuning knob 236 and fine-tuning grip 238 configured to allow an operator to make small adjustments in a limited range to achieve optimal tension in the whole blood bag 3.

The blood bag neck holder 220 is affixed to the frame 210 by the blood bag neck holder extruded component 213, and the blood bag tensioner 230 is affixed to the frame by the blood bag tensioner extruded component 214. Thus, the whole blood bag 3 is secured to the frame 210 between the front and back plates 242, 244 of the isolation device 200 with stability during all subsequent processing steps.

As shown in FIG. 4A, the plasma satellite bag 4 is placed towards the top of the isolation device 200 by hanging it onto the plasma hook 202. The plasma satellite bag 4 is positioned at an elevation above the whole blood bag 3 in order to prevent the flow of the blood sample into the plasma satellite bag 4 after the whole blood breakaway cannula 6 is broken. The whole blood breakaway cannula 6 is broken at the beginning of the isolation process, when the whole blood bag 3 is placed on the isolation device 200, ensuring that there is empty space in the whole blood bag 3 and a free gateway leading to the satellite plasma bag 4 for the plasma to move upwards during the following steps. In some embodiments, the cannula 6 is broken only if the sample is larger than a minimum threshold such as 100 ml.

The red blood cell bag 5 is positioned on an adjacent surface to the isolation device 200, such as a lab bench. The red blood cell bag 5 remains separate and isolated from the bags on the isolation device until the breakage of the red blood cell breakaway cannula 10 at a later step of the isolation process.

A second step includes preparation of the isolation device 200 for isolating the blood sample components using the isolation plate arrangement 240. The process can be standardized for samples of all volumes. Referring to FIG. 4F, the front plate 242 is mounted into place by aligning alignment holes 241a-241d therein with corresponding alignment rods 243a-243d extending from a back plate 244 of the isolation plate arrangement 240. The alignment rods 243a-243d are coupled to the back plate 244 in a stable manner and substantially parallel to each other, such that the front plate 242 and the back plate 244 are aligned exactly parallel to each other during the isolation process.

In some embodiments, the back plate 244 is affixed to the isolation device 200 such that it cannot be removed and can only move vertically by rotation of the elevation adjustment knobs 248a, 248b, which translate the rotation by an axle 249 through a support plate 253 to a pair of gears 251 which are operably coupled to a pair of geared tracks 252 that have a toothed surface for translating rotational motions of the gears 251 to vertical motion. The geared tracks 252 are affixed to the extruded components 211, 212 of the frame 210. Spacers 254 are then moved up and positioned between the front plate 242 and the back plate 244.

During use, an operator rotates torque-limited pressure knobs 246a, 246b in a clockwise direction, effectively screwing them onto the threaded bolts 245a, 245b extending from the back plate 244 toward the front plate 242. The threaded bolts 245a, 245b may be M8 threaded bolts which provide further alignment by the bolt holes 241e, 241f of the front plate 242. This action moves the front plate 242 towards the back plate 244. The front plate 242 moves slowly for a total distance of 15 mm in 20 seconds, therefore applying a small, gradual force on the whole blood bag 3, which is positioned between the front plate 242 and the back plate 244. A pair of ruled horizontal surfaces 250a, 250b on an upper surface of the isolation device 200 indicate to the operator how far the front plate 242 has traveled toward the back plate 244. The action moves the isolation device 200 from an open configuration of FIG. 4A towards a closed configuration. The torque-limited pressure knobs 246a, 246b stop rotating and the front plate 242 therefore stops moving when the front plate 242 comes into contact with the spacers 254, thus achieving a specific, predetermined distance between the front plate 242 and the back plate 244. The spacers 254 ensure a consistent, predetermined sample volume between the plates, regardless of the total volume of the sample, which then ensures uniformity in the following steps.

In a third step, the front and back plates 242, 244 are adjusted to commence the isolation process. The operator rotates the elevation adjustment knobs 248a, 248b to move both plates 242, 244 vertically to marked positions relative to the buffy coat line 33 as described below. In the illustrated embodiment, the operator has a clear vision of the blood components, including the buffy coat line 33, inside the whole blood bag 3 through the openings 247a of the front plate 242 and an activated light source which is attached on the back of the back plate 244 and penetrates to the front plate 242 by the openings 247b of the back plate 244. The vertical adjustment of the plates 242, 244 with respect to the buffy coat line 33 is carried out based on the values specified in a reference chart. A non-limiting example of a reference chart is provided below in Table 1.

Based on the values in the reference chart, by utilizing the corresponding markers 247c indicated on the number scale next to the see through openings 247a on the front plate 242 (the indexing numbers next to each of the see through openings 247a), the operator can position the plates precisely and with high repeatability to isolate the buffy coat and/or the predetermined plasma volume with a high degree of accuracy and with reduced or eliminated uncertainty. The whole blood bag 3 remains in a fixed, stable position during this process between the two plates 242, 244. The isolation device enables the application of the same method for the isolation of any segment of the umbilical cord blood by using different reference points.

TABLE 1

| | |
|---|---|
| For isolation of buffy coat | Position the buffy coat line at |
| without any plasma: | marker 9 |
| For isolation of buffy coat with | Position the buffy coat line at |
| 1 mL of plasma: | marker 9.5 |
| For isolation of buffy coat with | Position the buffy coat line at |
| 3 mL of plasma: | marker 10 |

Additionally, in some embodiments, the isolation device 200 utilizes a red blood cell concentrate (RBC) reference table for instances where the initial volume exceeds a specified threshold. In such cases, the RBC reference table determines the volume of the red blood cell component 32 that must be isolated to achieve the desired volume reduction in the whole blood bag 3. The RBC reference table may be used to determine a start point marking at which the buffy coat line is positioned prior to RBC isolation and an end point marking to which the buffy coat line reaches after the red blood cell component 32 has flowed to the satellite red blood cell bag 5. The RBC reference table relates the sample volume, the starting point marking for the buffy coat line, and the end point marking once the red blood cell component has been removed. To perform the RBC volume reduction procedure, an additional pair of spacers identical in size to spacers 254 is inserted through rods 243b and 243d, adjacent to the spacers 254. These spacers double the gap between the front plate and the back plate, enabling the operator to reduce the red blood cell count prior to isolating the buffy coat. Subsequently, after the red blood cell volume reduction is completed, the additional pair of spacers is removed, and the operator continues with the plasma reference table procedure described herein, with the start point marking for the plasma isolation being the volume of the sample after subtracting the volume of the red blood cell component 32 transferred to the RBC satellite bag.

Further, the reference charts can be implemented in a software such as a non-transitory computer readable medium which can be installed and used with a computer. In this case the operator will enter the initial blood volume of whole blood bag 3 and the desired plasma volume to be included in the final isolated blood component, and will then automatically receive instructions as to where to position the buffy coat line 33 on the scale of the openings 247a. This software can be installed on an independent computer which is used to assist the operator, or installed on the automated isolation plate arrangement's software (which is described further below in the description of FIG. 5).

In a third step of the isolation process, isolation of the desired blood components occurs. Upon achieving the desired positioning of the plates 242, 244 relative to the buffy coat line 33 in the whole blood bag 3, the spacers 254 are removed. The torque limited pressure knobs 246a, 246b are then rotated and screwed further onto the threaded bolts 245a, 245b by the operator, which causes the front plate 242 to move towards the back plate 244, further pressing the whole blood bag 3 which rests in a fixed position between the two plates 242, 244. By slowly pressing the whole blood bag 3 between the two plates 242, 244, the blood components inside the whole blood bag 3 are moved upwards towards the top of the whole blood bag 3. The whole blood bag cannula 6 is already broken, as described above in the first step, ensuring that there is empty space and a free gateway for the plasma to move upwards into the plasma bag 4. The pressing occurs gradually as the front plate 242 moves toward the back plate 244 maintaining the centrifuged blood components as separated, preventing any mixing.

The torque-limited pressure knobs 246a, 246b are equipped with press control, ensuring that once tightened to a specific force, the control is activated. This feature enables the operator to tighten the plates until a specified force is exerted, which prevents further rotation and effectively safeguards the whole blood bag 3 against potential damage. It also guides the operator in applying the necessary force to isolate the blood components. The movement halts when an isolation clamping system 255 of the front plate 242 and the back plate 244 come into contact with each other.

To detail use of the reference tables, once the whole blood bag 3 is positioned on the blood bag neck holder 220 and blood bag tensioner 230, the spacers 254 and the red blood cell reduction spacers are inserted on rods 243b and 243d of the isolation device 200. The front plate 242 is positioned on and pushed along the alignment rods toward the back plate 244 and the pressure knobs 246 are attached to the rods 243. The knobs 246 are rotated until the front plate 242 contacts the spacers 254. The elevation knobs 248 are adjusted until the buffy coat line 33 of the blood bag 3 is aligned with the marking along the scale 247c identified in the RBC reference table for the volume size. The tubing 18 is temporarily sealed with a clamp, and the cannula 6 is broken to allow the red blood cells to flow to the red blood cell bag 5 by gravity until the buffy coat line 33 reaches the RBC marking along the scale 247c determined from the RBC reference table.

Once the specified volume of red blood cell component 32 is transferred to the red blood cell bag 5, the RBC spacers are removed and the elevation knobs 248 are again adjusted until the buffy coat line 33 of the whole blood bag 3 is aligned with the marking along the scale 247c, as indicated in the plasma reference table. The spacers 254 are then disengaged, and the pressure knobs 246 are rotated to move the front plate 242 toward the back plate 244. The satellite plasma bag 4 is brought to an elevation below the whole blood bag 3, and the plasma transfers by gravity from the whole blood bag 3 to the plasma bag 4. Once the plasma transfer is complete, the tubing 16, 18 can be sealed to isolate the blood component in the whole blood bag 3.

Each of the front plate 242 and the back plate 244 may have a curved inner surface positioned opposite to each other. In the illustrated embodiment, the clamping system 255 is a male-female clamping system, with male clamping surfaces on the inner edge of the back plate 244 and female clamping surfaces on the inner edge of the front plate 242. By employing the male-female clamping system, the inner edges of the plates 242, 244 achieve a secure lock, with the curved surfaces forming a cavity that precisely, accurately, and efficiently isolates the desired volume and type of blood components inside the whole blood bag 3. The isolation is achieved precisely, with the total volume of the isolated blood component being equal to the total volume of the cavity formed by the two plates. The overall volume of the cavity can be adjusted, either increased or decreased according to the procedural needs. This adjustment should be performed before utilizing the apparatus for processing.

In a fourth step of the isolation process, the isolated blood components are transferred to their corresponding compartments in the blood processing set 20. Once the desired blood components are secured between the plates 242, 244, the operator initiates the transfer of the isolated blood sample from the top compartment of the whole blood bag 3 into the satellite plasma bag 4. Following this, tubing 16 is sealed, permanently isolating the blood component in the satellite plasma bag 4. The same procedure is replicated to isolate a further blood component, such as the red blood cells, in the lower locked compartment of the whole blood bag 3 by breaking the red blood cell cannula 10 and transferring the blood sample into the satellite red blood cell bag 5. Subsequently, tubing 18 is sealed, permanently isolating the blood component in the red blood cell bag 5. By sealing both tubing 18 and tubing 16, the remaining blood components, such as the buffy coat, located in the whole blood bag 3 is also effectively isolated.

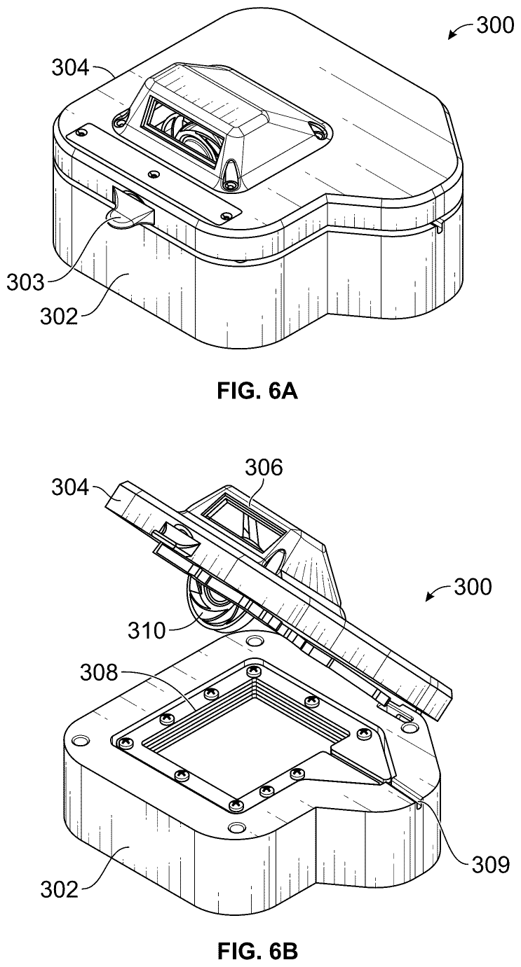

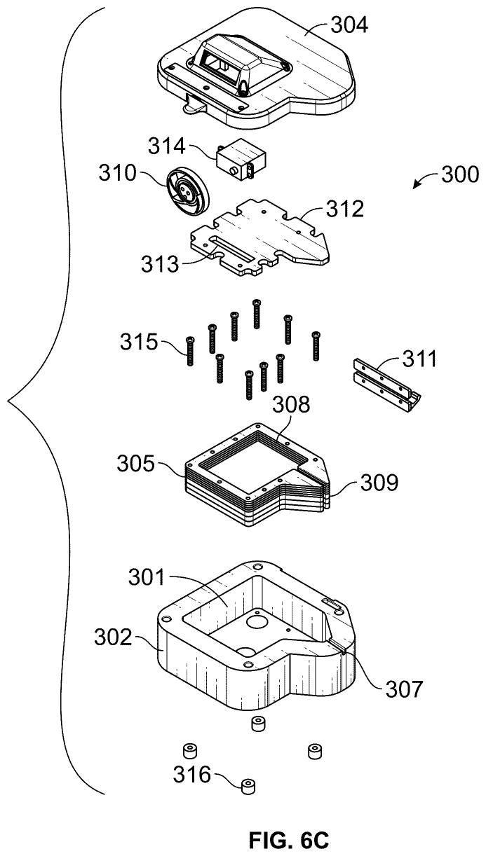

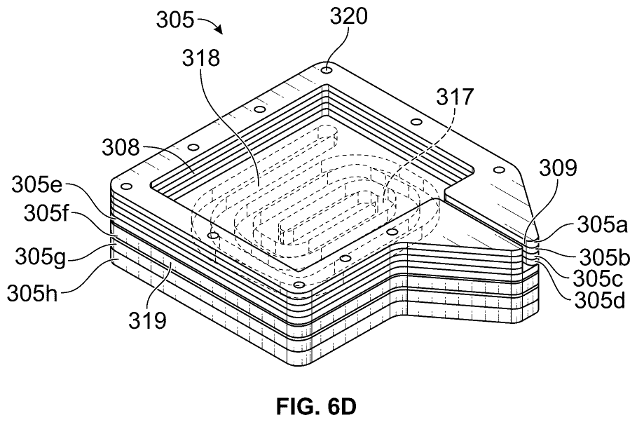

In a fifth step of the isolation process, the procedure is concluded by unscrewing the torque-limited pressure knobs 246a, 246b to allow for removal of the front plate 242. The desired blood components, typically the buffy coat and a volume of plasma, are transferred from the whole blood bag 3 to a cryobag for manual insertion to the cryopreparation device as described below with respect to FIGS. 6A to 6D.

FIG. 5 illustrates an example of an automated isolation plate arrangement 260, which is an automated version of the isolation plate arrangement 240 of FIG. 4F and which may be mounted on the frame 210 in a similar manner to the manual version 240. One or more computers, controllers, processors or control units 270 is used to control the automations of this embodiment through the use of rotors, motors, or other devices configured to move components such as the front plate 268 and the back plate 269. A reference chart(s), such as the reference chart of Table 1, in this embodiment is designed in a software which is configured to be installed and used with the computer of the automated device. In one embodiment, the operator enters the initial blood volume in whole blood bag 3 and the desired plasma volume to be included in the final isolated buffy coat, and the isolation plate arrangement 240 automatically determines the location and positioning of movable front and back plates 268, relative to the buffy coat line 33.

This automated version of the isolation arrangement 260 operates automatically, eliminating the need for manual knob adjustments by the operator. Automation is achieved through the integration of electric motors and actuators that respond to commands given from the software through the control unit or computer. Certain user data inputs may be utilized by the automated isolation arrangement 260, including (i) the total volume of the sample; (ii) the desired volume to be isolated; and (iii) the type of blood component or combination of plasma and buffy coat to be isolated.

In the illustrated embodiment, the automated isolation plate arrangement 260 analyzes the contents of the whole blood bag 3 via an optical sensor 267, which is positioned behind the back plate 269. An optical sensor slit 265 in the plate 269 allows the optical sensor 267 to view the blood bag 3. The optical sensor 267 is connected to an interface board, which includes instructions for calculating and commanding rotors, motors, and other such devices to move the movable plates 268 towards each other and apply a predetermined force on the whole blood bag 3. While an optical sensor 267 is used, the skilled artisan would understand that other sensors may be used in the alternative.

Once the portion of the whole blood bag 3 between the movable plates 268 reaches the desired volume, the plates stop moving. The optical sensor 267 detects color measurements of the blood components of the whole blood bag 3, and the controller is configured to identify different regions of the blood components based on the color measurements. The controller then adjusts the position of the plates 268, 269 along the vertical axis, based on the location of the buffy coat line 33, similar to the manual operation.

After the movable plates 268, 269 are at the desired position, the movable clamps 262a, 262b, 266a, 266b are moved toward and pressed against each other though clamp slits in the movable plates 268, 269 via linear or clamp actuators 264. The targeted blood components inside the whole blood bag 3 are isolated. The speed and force exerted by the automated isolation plate arrangement 260 is equal to or substantially equal to the amounts used in the manual isolation plate arrangement 240.

While the automated isolation plate arrangement 260 adheres to the same blood component isolation principles as the manual plate arrangement 240, the configuration of the plates differs. Each movable plate 268, 269, positioned exactly opposite to its counterpart, lacks alignment rods or spacers, with the software monitoring and guiding their movements. The flat surfaces of the movable plates 268, along with the accuracy of the optical sensor 267, allows them to stop vertically with an exceptional level of precision. This ensures a highly accurate and reliable preparation of blood component volume to be isolated. Consequently, the movable clamps 262a, 262b, 266a, 266b achieve a high-precision isolation of blood components.

Regarding the automation of the isolation plate arrangement 260, the computers, controllers, processors or control units 270 described herein may be adapted run a variety of application programs, access and store data, including accessing and storing data in associated databases, and enable one or more interactions via user devices. Typically, the one or more controllers are implemented by one or more programmable data processing devices. The hardware elements, operating systems, and programming languages of such devices are conventional in nature, and it is presumed that those skilled in the art are adequately familiar therewith. For example, the one or more controllers may be a PC-based implementation of a central control processing system utilizing a central processing unit (CPU), memories and an interconnect bus. The CPU may contain a single microprocessor, or it may contain a plurality of microprocessors for configuring the CPU as a multi-processor system. The memories include a main memory, such as a dynamic random access memory (DRAM) and cache, as well as a read only memory, such as a PROM, EPROM, FLASH-EPROM, or the like. The system may also include any form of volatile or non-volatile memory. In operation, the main memory stores at least portions of instructions for execution by the CPU and data for processing in accord with the executed instructions.

The controller may also include one or more input/output interfaces for communications with one or more processing systems. Although not shown, one or more such interfaces may enable communications via a network, e.g., to enable sending and receiving instructions electronically. The communication links may be wired or wireless.

The one or more controllers 270 may further include appropriate input/output ports for interconnection with one or more output displays (e.g., monitors, printers, touchscreen, motion-sensing input device, etc.) and one or more input mechanisms (e.g., keyboard, mouse, voice, touch, bioelectric devices, magnetic reader, RFID reader, barcode reader, touchscreen, motion-sensing input device, etc.) serving as one or more user interfaces for the controller. For example, the one or more controllers may include a graphics subsystem to drive the output display. The links of the peripherals to the system may be wired connections or use wireless communications.

Although summarized above as a PC-type implementation, those skilled in the art will recognize that the one or more controllers also encompasses systems such as host computers, servers, workstations, network terminals, and the like. The use of the term controller is intended to represent a broad category of components that are well known in the art.

As with the manual isolation plate device 200, at this point the desired blood components, typically the buffy coat and a volume of plasma, are transferred from the whole blood bag 3 to a cryobag for manual insertion to the cryopreparation device as described below with respect to FIGS. 6A to 6D.

FIGS. 6A to 6D illustrate a cryopreparation device 300 of the blood processing system 50. The cryopreparation device 300 maintains a steady temperature of 4° C.±0.1° C. for the sample in the cryobag for administering the cryoprotectant, such as dimethylsulfoxide (DMSO). Administering DMSO at this specific temperature minimizes toxicity and protects the sample of blood component. At 4° C., the blood sample is at the optimal lowest temperature before cryopreservation, preventing crystal formation or cellular damage, and reducing the toxicity of DMSO. The cryopreparation device 300 is a device that reduces and maintains the temperature of the cryobag, including the contents from the whole blood bag 3 received at the end of the isolation process, at a constant 4° C.±0.1° C. Additionally, the device provides proper mixing of the DMSO with the sample during its administration.

Referring to FIGS. 6A and 6B, the cryopreparation device 300 comprises a thick hollow plastic base 302 and cover 304 coupled via a hinge 311 that allows the cryopreparation device to switch between a closed configuration (such as in FIG. 6A) and an open configuration (such as in FIG. 6B). A handle 303 may be provided to aid a user in switching between configurations.

The base 302 and cover 304 contain aluminum surfaces 312 and cavity stack 305 that are actively liquid cooled by routing cooled liquid through a serpentine or other fluid pathway 317 (see FIG. 6D) built into a cavity stack 305 which nests into the base 302. In the illustrated example, the cavity stack 305 is made of eight different cavity plates 305a-305h, although any number of cavity plates may be used as desired. Each of the top four cavity plates 305a-305d has a large cutout which collectively form a compartment 308 in which the cryobag is positioned. The next cavity plate 305e provides a flat surface for the cryobag to rest upon. The remaining cavity plates 305f-305h house fluid pathways for the coolant to flow through and ensure proper heat transfer for the sample (or sample in the cryobag) to reach 4° C.

Further, the cover 304 includes an aluminum plate that, when closed, contacts the aluminum of the cryobag compartment 308. This design facilitates the transfer of temperature from the aluminum cavity stack 205 and compartment 308 to the cover 304. Additionally, the cover 304 directly contacts the top surface of the cryobag when closed, enabling the transfer of temperature to the bag. This configuration ensures even temperature distribution throughout the cryobag from all sides.

A plurality of holes 320 extend through a perimeter of the cavity stack 305 to form bores and allow the cavity stack 305 to be coupled to the base 302 by fasteners 315 and rest in the cavity stack compartment 301. A plurality of feet may be attached to some or all of the fasteners to provide a higher-friction contact to the surface that the cryopreparation device rests upon.

The base 302 and cover 304 provide excellent thermal sealing from the environment. The aluminum plates 312 act as an excellent temperature conductor, which allows the biological material in the cryobags to reach 4° C. and maintain this temperature during the administration of the cryoprotectant. The contact between the aluminum plates 312 and the cryobag promotes efficient heat transfer between the cryobag and the cryopreparation device 300, such configuration ensuring even temperature distribution throughout the cryobag from all sides of the cryopreparation device 300. The liquid coolant, maintained at a monitored temperature below 4° C., flows through the fluid pathways 317 until the compartment 308 reaches the target temperature of 4° C., at which point the circulation stops. The compartment's insulating material of the base 302 and cover 304 maintains this temperature for a sufficient period. If the probes in the compartment 308 detect a temperature higher than 4.0° C. (to one decimal place), the liquid coolant circulation resumes until the temperature drops back to 3.9° C.

A cryopreparation system may be implemented wherein multiple cryopreparation devices 300 which are independently operated, and temperature controlled via a specialized automated electronic controller are provided. Each cryopreparation device 300 is equipped with its own digital thermometer, allowing the current temperature to be displayed individually. Each cryopreparation device 300 is equipped with a motor 314 which is constantly pulsating a mixer 310 (which is visible through a window 306 and sits in a plate aperture 313 of the aluminum plate 312), causing the blood sample in the cryobags to be mixed smoothly with the cryoprotectant, ensuring even distribution of the cryoprotectant solution. This independent temperature monitoring and control of each cryopreparation device 300 enables continuous uninterrupted operation by the end user of multiple cryobags.

A tube insertion port 307, with access to the chamber 308 by tube channel 309 cut into the top four cavity plates 305a-305d, ensures that the DMSO reaches a temperature of 4° C. before administration to the sample. The DMSO is typically kept in a syringe pump at laboratory ambient temperature (around 20° C.) and, due to the slow administration rate, the temperature rises above 4° C. The DMSO is applied at a slow, steady rate of about 0.3 ml/min, allowing sufficient time within the tube insertion port to achieve the ideal temperature prior to administration. Additional cryopreparation devices 300 can be easily added to the system, helping prevent any bottleneck issues and eliminating the need to purchase additional devices for this purpose.

It should be understood that reference is made to a blood processing system. This blood processing system includes the blood bag housing device 100, the isolation device 200, and the cryopreparation device 300, and any components or alternative embodiments thereof. The blood processing system is the combination of these devices along with the methods of using them to effectively process a blood sample from the time it is drawn to the time it is ready for testing or to be placed in long term cryostorage.

It should be noted that various changes and modifications to the presently preferred embodiments described herein will be apparent to those skilled in the art. Such changes and modifications may be made without departing from the spirit and scope of the present invention and without diminishing its attendant advantages.

What is claimed is:

1. A blood bag housing device for use in a centrifuge, the blood bag housing device comprising:
a main body including a front portion and a back portion spaced from the front portion by a cavity, wherein the back portion includes a curved surface at a top of the main body, and wherein a bottom of the main body includes one or more pathways;
a retainer cover removably couplable to the main body;
wherein the cavity is configured to receive a blood bag; and
wherein the main body is configured to seat in a bucket of the centrifuge.

2. The blood bag housing device of claim 1, wherein a back pocket in the back portion is configured to house at least one tubing coupled to a top of the blood bag when the retainer cover is affixed to the back pocket, and
wherein the front pocket is configured to house at least one tubing coupled to the bottom of the blood bag.

3. The blood bag housing device of claim 2, wherein at least one of the one or more pathways is configured to receive at least one of the at least one tubing housed in the back pocket.

4. The blood bag housing device of claim 1, wherein the blood bag housing device is configured to be submerged in water during centrifugation.

5. The blood bag housing device of claim 1, further comprising an aperture in the retainer cover.

6. The blood bag housing device of claim 1, wherein the front pocket is further configured to house at least one satellite bag.

7. The blood bag housing device of claim 1, wherein the retainer cover comprises at least one tabbed surface that is received by a slotted opening in the main body.

8. An isolation device of a blood processing system, the isolation device comprising:
a frame;
a blood bag neck holder mounted to the frame, the blood bag neck holder comprising an upper holder rod configured to receive a top opening of a blood bag;
a blood bag tensioner secured to the frame, the blood bag tensioner comprising a lower holder rod configured to receive a bottom opening of a blood bag; and
an isolation plate arrangement mounted to the frame and comprising a front plate and a back plate movable relative to each other,
wherein the isolation plate arrangement is configured to at least partially house the blood bag between the front plate and the back plate when the blood bag is secured to the blood bag neck holder and the blood bag tensioner.

9. The isolation device of claim 8, wherein the back plate is secured to the frame and includes a threaded bolt extending forwardly from the back plate and through an opening in the front plate, and wherein the isolation plate arrangement includes a pressure knob that engages the threaded bolt and moves the front plate relative to the back plate.

10. The isolation device of claim 9, wherein the back plate further comprises at least one spacer configured to provide a consistent spacing between the front plate and the back plate as the pressure knob is adjusted.

11. The isolation device of claim 8, wherein the front plate further comprises a plurality of alignment holes disposed around a perimeter of the front plate; and wherein the back plate further comprises a plurality of alignment rods configured to slidably engage the alignment holes to align the front plate and the back plate during operation of the isolation device.

12. The isolation device of claim 8, wherein the back plate is secured to a support plate through which an axle extends, a gear and a vertical adjustment knob are affixed to the axle adjacent the support plate, wherein the gear engages a geared track attached to the frame such that rotation of the gear translates to a vertical displacement of the back plate.

13. The isolation device of claim 8, wherein the isolation plate arrangement comprises:
one or more rotors configured to move at least one of the front plate and the back plate;
at least one clamp actuator configured to move a clamp toward one of the front plate and the back plate such that the one of the front plate and the back plate are secured in position; and
an optical sensor configured to detect blood components in the blood bag disposed between the front plate and the back plate,
wherein the optical sensor and at least one rotor are controlled by a control unit.

14. A cryopreparation device of a blood processing system, the cryopreparation device comprising:
a base including a cavity stack compartment;
a cover;
a cavity stack compartment in an interior of the base; and
a cavity stack disposed in the cavity stack compartment, the cavity stack comprising:
a plurality of cavity plates configured to be stacked within the cavity stack compartment to seat a cryobag,
wherein a first cavity plate on a top side of the cavity stack comprises a compartment configured to enclose the cryobag and at least one tube channel configured to receive a cryoprotectant tube, and
wherein a second cavity plate disposed below the first cavity plate comprises a fluid pathway configured to allow the flow of a cooling fluid in the interior of the base.

15. The cryopreparation device of claim 14, wherein the cryopreparation device is configured to maintain a temperature of the cryobag at about 4° C.

16. The cryopreparation device of claim 14, further comprising a temperature sensor configured to detect a temperature of the cryobag, and wherein the cryopreparation device is configured to start or stop the circulation of cooling fluid in the interior of the base if the temperature of the cryobag is sensed to be above or below about 4° C.

17. The cryopreservation device of claim 14, further comprising a motor configured to pulsate fluid inside the cryobag.

18. The cryopreservation device of claim 17, wherein a mixer is coupled to the motor and configured to evenly mix fluid inside the cryobag.

19. The cryopreservation device of claim 14, further comprising an aluminum plate disposed on an interior of the cover.

20. The cryopreservation device of claim 14, wherein the cavity stack is affixed to the base by a plurality of fasteners through a plurality of holes disposed around a perimeter of the cavity stack and a perimeter of the cavity stack compartment.
